(19) United States
(12) Patent Application Publication
(10) Pub. No.: US 2015/0051882 A1
(43) Pub. Date: Feb. 19, 2015

(54) Artificially Simulating Emissions of a Chemical Compound

(71) Applicant: TECHNOLOGY S.G., LP, West Conshohocken, PA (US)

(72) Inventors: Eric L. CANFIELD, Exton, PA (US); Robert P. ALSTON, Exton, PA (US); Thomas M. DYKSTRA, Alachua, FL (US)

(73) Assignee: TECHNOLOGY S.G., LP, West Conshohocken, PA (US)

(21) Appl. No.: 14/014,065

(22) Filed: Aug. 29, 2013

Related U.S. Application Data
(63) Continuation-in-part of application No. 13/969,001, filed on Aug. 16, 2013, now abandoned.

(51) Int. Cl. A01K 67/033; G06F 19/00

(52) U.S. Cl. CPC A01K 67/033; G06F 19/704
USPC 703/2; 119/6.5; 703/12

(57) ABSTRACT

An apparatus is provided for causing a behavioral response in an insect species. The apparatus may include a housing, a radiating emitter, a directing apparatus, and a power source coupled to the radiating emitter. The radiating emitter may be configured to emit radiation at one or more wavelengths simulating an emission spectrum of a chemical compound of interest that may cause a behavioral response in the insect species. The directing apparatus disposed within the housing may be configured to control a direction of the emitted radiation and the power source may be configured to control an intensity of the emitted radiation.

### CROSS REFERENCE TO RELATED APPLICATION

The present application is a continuation-in-part of U.S. Non-Provisional application Ser. No. 13/969,001, filed Aug. 16, 2013, which is hereby incorporated by reference in its entirety.

### FIELD

Embodiments of the present invention are directed to artificially simulating emissions of a chemical compound of interest (CCI) for affecting behavior of insect species.

### BACKGROUND

Charles Valentine Riley, former chief of the Entomological Commission and author of Insect Life suggested that insects might sense subtle vibrations to which we are blind [Insect Life, vol. 7, pp. 33-41 (1894)]. Jean Henri Fabre was the next to publish his thoughts in “The Insect World of J. Henri Fabre” [E. W. Teale (ed.), 191 pp. (1913)] that perhaps moths are tuned to certain electromagnetic (EM) frequencies, while Eugene Marais speculated in “The Soul of the White Ant” [Methuen and Co., London, 184 pp. (1937)] that termites might do the same.

EM frequencies are known to be picked up by appropriately structured antennae.

However, no one looked for them on insects until 1948 when Grant, an electrical engineer, was the first to publish a paper indicating a detector for this EM radiation on insect antennae and later found further support from Laithwaite [Proc. Royal Soc. Queensland, vol. 60, no. 8, pp. 89-98 (1948); Entomologist vol. 93, no. 1165, pp. 113-177 (1960); Entomologist vol. 93, no. 1166, pp. 133-137 (1960)]. Miles and Beck hypothesized that certain olfactory receptors are indeed radiation receptors, based upon their experiments with honeybees and the bees' attraction toward an enclosed box containing honey [Proc. Natl. Acad. Sci., vol. 35, pp. 292-310 (1949)]. This box was equipped with an infrared transparent window.

Evidence that insect antennae are well-equipped to be radiation detectors came primarily from Callahan [Misc. Public. Entom. Soc. Amer., vol. 5, no. 7, pp. 315-347 (1967)] and that some of these antennae respond unequivocally to radiation sources was supported by Evans [Nature, vol. 202, p. 211 (1964)] and Bruce [Ann. Entomol. Soc. Am., vol. 64, pp. 925-931 (1971)]. At this point, the radiation detectors were established.

Smith and colleagues have shown a remarkable sensitivity among insects to low-intensity radiation, which suggests that insects may have developed systems to detect low intensity levels with which we are still unfamiliar [Science, vol. 140, pp. 805-806, (1963)]. A very important feature of these biological effects is that they are often produced by fields of extremely low intensities. The effects of multiple exposures on the organism are sometimes cumulative. Exposure to strong fields usually leads to adaptation to subsequent exposures, whereas exposure to weak fields leads to progressively greater changes in the organism [“Electromagnetic Fields and Life,”, Plenum, New York-London, 336 pp., (1970)]. This naturally suggests that organisms have systems which are especially sensitive to EM fields and as yet have no analogs in man.

W. H. Whitcomb, his student, J. C. Nickerson, and Callahan collaborated on a short research project involving a worker ant, Conomyrma insana (Buckley), and infrared emissions [Physiol. Chem. & Physics, vol. 14, pp. 139-144 (1982)]. They found that the worker ant was attracted to the far-infrared emissions from wax and petroleum candles. Various species of moth and larvae were subjected to infrared radiation with wavelengths in the 1-30 μm (micrometer) wavelength band [J. Ga. Ent. Soc., vol. 1, pp. 6-14 (1966)]. High intensity infrared radiation focused into the eye killed moths in an average of 60 sec. at 120° F. Low intensity infrared focused on the antenna or eye elicited flight, antennal responses, or sexual responses, at 85 to 92° F. Low intensity infrared at 92° F. focused on the simple eyes (ocelli) of larvae elicited fecal pellet deposition, searching, and head scanning. All of these responses were repeatable and then became conveniently predictable.

Some examples of insects responding to radiation are described here. Fifth instar corn earworm larvae were subjected to the radiation for 15 to 40 sec., before becoming active, which consisted of depositing a fecal pellet, chewing with their mandibles, and moving toward the IR source while scanning it with the head. Noctuid adults responded by vibrating their antennae immediately. The coiled proboscis immediately went into a frenzy of movement. Curiously, sphingid moths and saturniid moths were much slower to respond with antennal movements. The four species of night-flying arctiid moths were by far the most sensitive. They all responded by curving the abdomen and feeling toward the source with both legs and antenna, and attempted to touch with their abdomen objects brought within their range (i.e. mating behavior). One second of high-intensity radiation of 1 to 30 μm elicited flight and sexual responses from these four species for a period of 10 to 20 minutes. Five to ten seconds of low intensity IR induced similar responses.

Evans showed that a buprestid beetle, Melanophila acuminata, possessed a distinct infrared sense organ located not on the antenna, but on the mesothorax adjacent to the coxal cavities [Nature, vol. 202, p. 211 (1964)]. Although several reports had found that insects, such as mosquitoes [Nature, vol. 184, pp. 1968-1969 (1959)], respond to infrared, this was the first reported insect infrared organ. The radiation used to elicit a response in Evans' studies was incoherent infrared radiation between 0.8 and 6.0 μm wavelengths, with a maximum sensitivity between 2.5 and 4 μm. Another arthropod, the female spiny rat mite, Laelaps echidnina, also responds to incoherent infrared radiation in the narrow band between 4.4 and 4.6 μm, as reported by Bruce [Ann. Entomol. Soc. Am., vol. 64, pp. 925-931 (1971)].

The Glagolewa-Arkadiewa “mass radiator” was used to further test the response of insects to pure EM radiation. The mass radiator was used to transmit far-IR radiation upon several insects in order to record their behavioral responses, if any, as reported by Callahan [Fla. Entomol., vol. 54, no. 2, pp.201-204 (1971)]. The responses were amazing. All insects tested responded to the mass radiator with antennal movement. Three mated corn earworm females were stimulated to oviposit within a few seconds of exposure to the radiation. Wasps immediately exhibited the antennal cleaning response, and fire ants responded by violent movement of the legs and antennae. Not a single response from any of the insects tested occurred when the antennae were cut off. One year later, Eldumiati and Levengood found extremely strong attractive responses of insects to far-IR radiation as well [J.Econ. Entomol. Vol. 65, pp. 291-292 (1972)].

Behavioral responses to broadband IR in the 1-15 μm range have been reported from three different orders of insects. They include the Lepidoptera, H. zea [Ann. Entomol. Soc. Am., vol. 58, pp. 746-756 (1965)], Diptera, Aedes aegypti [J. Econ. Entomol., vol. 61, pp. 36-37 (1968)] and a braconid wasp from the order Hymenoptera, Coeloides brunneri (Can. Entomol., vol. 104, pp. 1877-1881, (1972)].

Furthermore, the Red Imported Fire Ant, Solenopsis invicta, is attracted to electric fields and is able to distinguish between AC and DC fields [Environ. Entomol., vol. 21, no. 4, pp. 866-870 (1992)] and electrophysiological responses of some antennae on the scape and pedicel of several species of noctuid and saturniid moths were found to be stimulated by frequencies across the entire visible spectrum [J. Appl. Optics, vol. 7, pp. 1425-1430 (1968)].

Thus, insects have been shown to respond to short and long exposures of radiation, broadband IR, narrowband IR, low, mid, and far infrared radiation, visible frequencies, coherent radiation. They have also been shown to have well-studied radiation organs, and are able to differentiate between AC and DC fields. The ability to stimulate insect antennae with EM frequencies, or radiation, initiated a search for novel sources of radiation detection. The luminescence characteristics of semiochemicals, as well as other odorants and various chemical compounds, provided this radiation. Pheromones, which are just one type of semiochemical, exhibit powerful behavioral responses in insects and may serve to attract or confuse insects, thus disrupting their mating successfully.

Many pheromone traps have limitations in significantly reducing insect populations in a storage grain bin or warehouse unless the traps are used in very high densities. With respect to aerosol or lure deployment for agricultural control of insect species on farmland, it is an expensive proposition with numerous limitations. Inclement weather, high winds, and other factors adversely affect these control measures and often prevent a pest management program from succeeding. Additionally, the reduced longevity of the pheromone source in traps, as well as the expense of the pheromone itself, help contribute to these problems.

### BRIEF SUMMARY

Methods provided herein artificially simulate characteristics of chemical compounds that induce behavioral responses in an insect species. Also provided herein is an apparatus configured to reproduce characteristics of chemical compounds that induce behavioral responses in an insect species.

In an embodiment of the present invention, a method of artificially simulating a chemical compound emission includes identifying a chemical compound of interest, determining an infrared (IR) radiation absorption spectrum and/or a UV-Visible (UV-Vis) absorption spectrum of the chemical compound of interest, applying a Stokes shift to at least one absorption wavelength value of the absorption spectrum, and approximating an emission spectrum of the chemical compound of interest based on the applying. The determined absorption spectrum includes at least one absorption wavelength value and the approximating emission spectrum includes at least one emission wavelength value.

In another embodiment of the present invention, a method of artificially simulating an emission of a chemical compound of interest includes identifying a chemical compound of interest, modeling one or more ground states of the chemical compound of interest, and modeling one or more excited states of the chemical compound of interest based on the modeled one or more ground states. The method further includes producing a simulated UV-Vis emission spectrum based on a geometry optimization calculation of the modeled one or more excited states and/or a simulated IR emission spectrum of the chemical compound of interest based on a anharmonic frequency calculation of the modeled one or more ground states. The produced emission spectrum includes at least one emission wavelength value.

In another embodiment of the present invention, a method of artificially simulating an emission of a chemical compound of interest includes identifying a chemical compound of interest and empirically determining an emission spectrum of the chemical compound of interest through Fourier transform infrared (FTIR) spectroscopy.

In another embodiment of the present invention, an apparatus for causing a behavioral response in an insect species is provided. The apparatus includes a radiating emitter, a directing apparatus, and a power source coupled to the radiating emitter. The radiating emitter may be configured to emit radiation at one or more wavelengths simulating an emission spectrum of a chemical compound of interest that may cause a behavioral response in the insect species. The directing apparatus may be configured to direct the emitted radiation and the power source may be configured to control an intensity of the emitted radiation.

In another embodiment of the present invention, an apparatus for causing a behavioral response in an insect species is provided. The apparatus includes a radiating emitter and a power source coupled to the radiating emitter. The radiating emitter may be configured to emit radiation at one or more wavelengths simulating an emission spectrum of a chemical compound of interest that may cause a behavioral response in the insect species. The power source may be configured to control an intensity of the emitted radiation.

Further features and advantages, as well as the structure and operation, of various embodiments of the present invention are described in detail below with reference to the accompanying drawings. It is noted that the present invention is not limited to the specific embodiments described herein. Such embodiments are presented herein for illustrative purposes only. Additional embodiments will be apparent to persons skilled in the relevant art(s) based on the teachings contained herein.

### BRIEF DESCRIPTION OF THE DRAWINGS/FIGURES

The accompanying drawings, which are incorporated herein and form part of the specification, illustrate the present invention and, together with the description, further serve to explain the principles of the invention and to enable a person skilled in the pertinent art to make and use the invention. in the drawings, like reference numbers indicate identical or functionally similar elements. Additionally, the leftmost digit(s) of a reference number identifies the drawing in which the reference number first appears.

FIG. 1 illustrates a flowchart for a method of artificially simulating emission of a chemical compound of interest (CCI) according to an embodiment.

FIG. 2 illustrates a schematic of an example empirical infrared (IR) absorption spectrum of a CCI.

FIG. 3 illustrates a flowchart for a method of artificially simulating emission of a CCI according to an embodiment.

FIGS. 4, 5, and 6 illustrate flowcharts for a mathematical modeling of molecular states of a CCI according to embodiments.

FIG. 7 illustrates a schematic of an example simulated IR absorption spectrum of a CCI.

FIG. 8 illustrates a schematic of an example simulated excited state IR vibrational spectrum of a CCI.

FIG. 9 illustrates a flowchart for a method of artificially simulating emission of a CCI according to an embodiment.

FIG. 10 illustrates a flowchart for a method of empirically determining an emission spectrum of a CCI according to an embodiment.

FIGS. 11 and 12 illustrate schematic diagrams of apparatuses for causing a behavioral response in an insect species according to embodiments.

FIG. 13 illustrates a block diagram of a computer system in which embodiments of the present invention, or portions thereof, may be implemented.

The features and advantages of the present invention will become more apparent from the detailed description set forth below when taken in conjunction with the drawings.

### DETAILED DESCRIPTION

The following Detailed Description refers to accompanying drawings to illustrate one or more embodiments consistent with the present disclosure. The disclosed embodiment(s) merely exemplify the disclosure. References in the Detailed Description to “an example embodiment,” “an example of this embodiment,” etc., indicate that the embodiment(s) described may include a particular feature, device, or characteristic, but every embodiment may not necessarily include the particular feature, device, or characteristic. Moreover, such phrases are not necessarily referring to the same embodiment. Further, when a particular feature, device, or characteristic is described in connection with an embodiment, it is within the knowledge of those skilled in the relevant art(s) to effect such feature, device, or characteristic in connection with other embodiments whether or not explicitly described.

The embodiments described herein are provided for illustrative purposes, and are not limiting. Other embodiments are possible, and modifications can be made to the embodiments within the spirit and scope of the present disclosure. Therefore, the Detailed Description is not meant to limit the present disclosure. Rather, the scope of the present disclosure is defined only in accordance with the following claims and their equivalents.

Some embodiments of the disclosure may be implemented in hardware, firmware, software, or any combination thereof. Some embodiments of the disclosure can also be implemented as instructions stored on a machine-readable medium, which can be read and executed by one or more processors. A machine-readable medium can include any mechanism for storing or transmitting information in a form readable by a machine (e.g., a computing device). For example, a machine-readable medium can include non-transitory machine-readable mediums such as read only memory (ROM); random access memory (RAM); magnetic disk storage media; optical storage media; flash memory devices; and others. As another example, the machine-readable medium can include transitory machine-readable medium such as electrical, optical, acoustical, or other forms of propagated signals (e.g., carrier waves, infrared signals, digital signals, etc.). Further, firmware, software, routines, instructions can be described herein as performing certain actions. However, it should be appreciated that such descriptions are merely for convenience and that such actions in fact result from computing devices, processors, controllers, or other devices executing the firmware, software, routines, instructions, etc.

It is to be understood that the phraseology or terminology herein is for the purpose of description and not of limitation, such that the terminology or phraseology of the present specification is to be interpreted by those skilled in relevant art(s) in light of the teachings herein.

Embodiments of the present invention provide methods for artificially simulating a chemical compound of interest (CCI) induce behavioral responses in an insect species. Embodiments of the present invention also provide an apparatus for inducing behavioral responses in an insect species. Inducing behavioral responses may be in the form of producing attractive, repulsive, or chaotic movement responses in the insect species, as will be described further below.

In an embodiment, the CCI emits EM radiation such as, but not limited to, infrared (IR) radiation. The EM radiation may be emitted by a type of luminescence, for example, photoluminescence, chemiluminescence, electroluminescence, thermoluminescence, electroluminescence or any combination thereof, according to various examples of this embodiment. Luminescence is a phenomenon of light being emitted from a body by any process or method other than high temperature emission. For example, in photoluminescence, molecules of a material are excited by incoming EM radiation to produce or emit light. When the release of EM energy is immediate, or ceases upon the removal of the exciting EM radiation in photoluminescence, the material is said to be fluorescent. Fluorescence is a type of photoluminescence that occurs when energy (e.g., light) from an excitation energy source is absorbed by a body (or molecule) at one or more wavelengths and is re-emitted at one or more different wavelengths. The photonic emission is generally of a longer wavelength than the excitation source. In simplest terms, fluorescence occurs when a molecule that has been excited to a higher energy state relaxes and returns to its “ground” (or neutral) state, releasing one or more photons.

The CCI may have an emission spectrum that maps its luminescence response.

The emission spectrum can be recorded by fixing excitation wavelengths at one or more particular wavelengths, while the intensity of one or more emission wavelengths is scanned. The CCI may also have a particular absorption spectrum in addition to an emission spectrum. An absorption spectrum of a body is a plot of the absorption intensity of the fraction of incident radiation absorbed by that body as a function of wavelengths covering the electronic energy levels of the molecules in the body. Absorption spectra can be recorded for any absorbing material. The CCI may also have a characteristic excitation spectrum as a part of its absorption spectrum. In an embodiment, the excitation spectrum of the CCI is the same as its absorption spectrum; in another embodiment, the excitation spectrum is a subset of its absorption spectrum. For ease of discussion, references to the absorption spectrum in the below discussion are also applicable to the excitation spectrum.

The CCI used in embodiments of the present invention may include odorants and semiochemicals such as, but not limited to, pheromones, kairomones, allomones, and synomones. An excellent reference database of well-known semiochemicals and pheromones is “The Pherobase” (www.pherobase.com), which contains thousands of chemical compounds and relevant formulas. The luminescence characteristics of the CCI may result in the emission of EM radiation at various wavelengths in an emission spectrum. Such wavelengths may range from, for example, 300 nm to 30 μm. Such range of wavelengths includes ultraviolet (UV), visible light (Vis), and IR light. As used herein, UV light includes wavelengths from 10 nm to 400 nm with corresponding photon energies from 124 eV to 3.10 eV, Vis light includes wavelengths from 390 nm to 700 nm with corresponding photon energies from 3.18 eV to 1.77 eV, and IR light includes wavelengths from 700 nm to 30 μm with corresponding photon energies from 177 eV to 41.33 meV. The wavelengths in the emission spectrum are referred to herein as the emission wavelengths. These wavelengths may be detected by an insect of a species and cause a change in its behavior.

If an insect species is sensitive to the emission wavelengths, such as those insect species responsive to a specific semiochemical such as, but not limited to, a pheromone, there are several types of behavior that may result from the insect species' exposure to the emission wavelengths. A first type of behavior is an attraction behavior. If the emission wavelengths correspond to those produced by a sex or aggregation pheromone, an insect species that detects the emission wavelengths may be attracted or lured to the pheromone as if it were a mating signal or a call to aggregate, respectively. A second type of behavior is a repelling behavior. If the emission wavelengths are too strong or are representative of something the insect species would perceive as a threat, an insect species may be overwhelmed and repelled by the signal, or seek evasive action or cover in the event of a perceived threat. A third type of behavior is confusion or chaotic response, which results when the emission wavelengths disrupt the insect species from its normal behavior. When some insect species are exposed to certain emission wavelengths, their behavior is disrupted. The insect species may, for example, become abnormally active, using up their own energy resources such that they are unable to properly mate, or such that they die sooner than expected. The depletion of their energy resources may also produce unhealthy offspring, eventually resulting in an overall reduction in an insect population.

The behavior of different types of insect species may be affected by different emission wavelengths. These different wavelengths may include different luminescence wavelengths of the same CCI, or the different wavelengths may include luminescence wavelengths of multiple CCIs. Therefore, one CCI may be used as an attractant, repellent, or disruptive agent for different types of insect species. Also, the same insect species may exhibit one type of response for one CCI, and another type of response for a different CCI.

Several limitations have been found in insect traps or lures that use quantities of the physical CCL One limitation is the reduced efficacy of the CCI over a period of time.

The reduced efficacy may be due to a gradual decay of the CCI's luminescence with time and usage, and/or due to a reduction in effectiveness at increased distances from the CCI. To compensate for the reduced efficacy, larger numbers of traps may be used, making this method of pest control expensive. Another limitation is the inability to disable these CCI-based traps when not in use.

These limitations may be overcome by replacing the physical CCI with an apparatus that mimics the CCI. For example, embodiments discussed below artificially simulate the above-described emission characteristics of the CCI, such that an actual, physical CCI need not be used. For the sake of simplicity, the CCI will be referred to herein as emitting radiation by means of photoluminescence, in particular by means of fluorescence, in the following embodiments. It should be understood that the following embodiments may also be applied to CCI having any of the other luminescent characteristics discussed above.

### A METHOD OF ARTIFICIALLY SIMULATING EMISSION OF A CHEMICAL COMPOUND ACCORDING TO AN EMBODIMENT

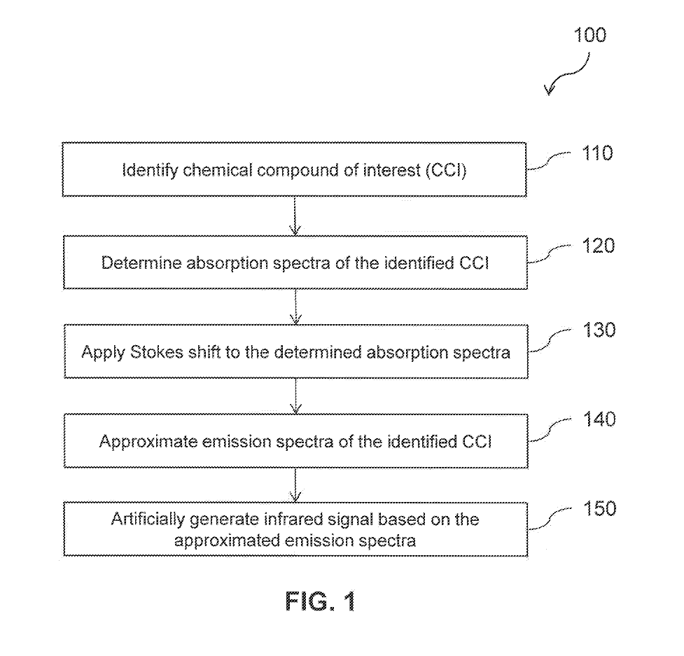

FIG. 1 illustrates a flowchart for a method of artificially simulating photonic emission of a CCI according to an embodiment.

In step 110, a CCI is identified. The identifying may be based on the type of insect species targeted for inducing one or more of the behavioral responses discussed above. An example is, the sex pheromone known to attract male Indian Meal Moths.

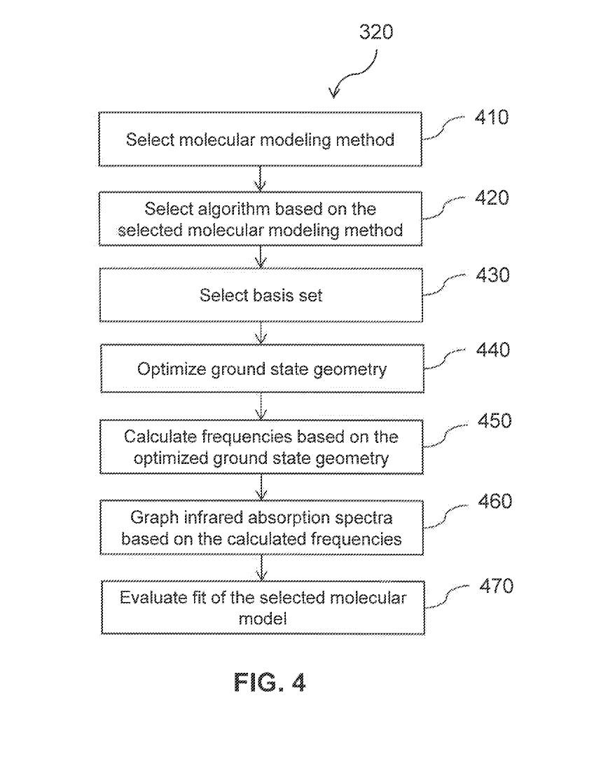

In step 120, an absorption spectrum of the identified CCI is determined. The absorption spectrum including IR wavelengths and/or UV-Vis wavelengths may be determined from a lookup table that may be accessed from a storage medium. In an embodiment, the lookup table stores predetermined absorption spectra of different CCIs. The predetermined absorption spectra may be obtained, for example, empirically or by mathematical modeling based on a model described below with reference to FIGS. 4 and 5.

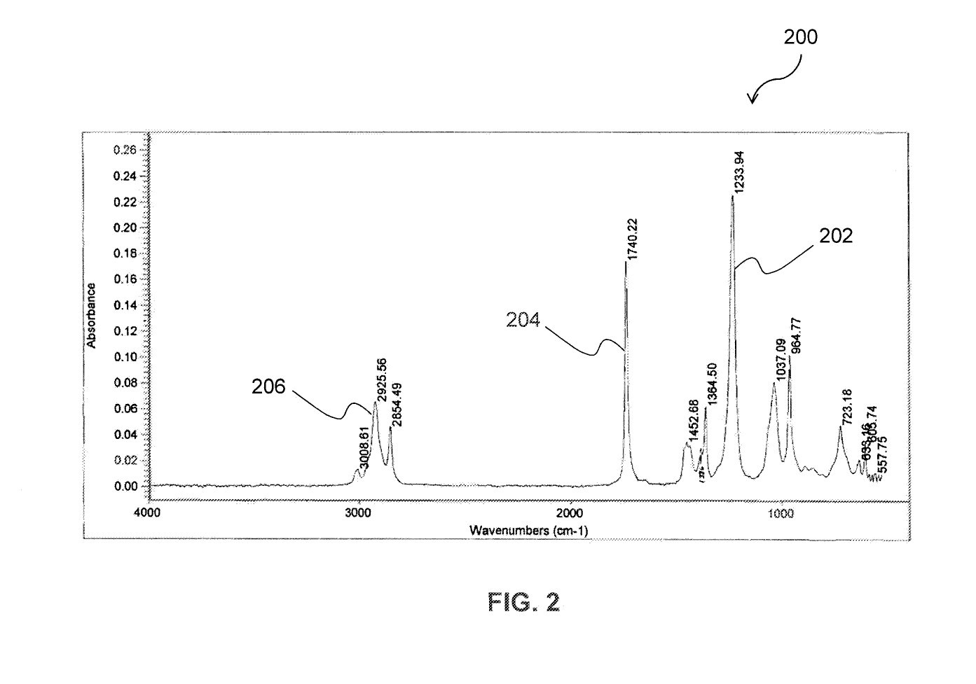

In an embodiment, the predetermined empirical absorption spectrum of the identified CCI is obtained by optical spectroscopy such as, but not limited to, attenuated total reflectance (ATR) FTIR spectroscopy. ATR-FTIR spectroscopy utilizes the phenomenon of total internal reflection. For ATR-FTIR spectroscopy, the CCI may be placed in close contact with an ATR crystal that is transparent in the IR frequency range. A radiation beam from an IR source may be passed through the ATR crystal, allowing the radiation beam to reflect within the ATR crystal several times. The reflecting radiation beam may penetrate a few nanometers into the closely-placed CCI. This penetration may cause the CCI to absorb a portion of the reflecting radiation beam and consequently, the reflecting radiation beam may lose energy at the wavelengths absorbed by the CCI. The resultant attenuated radiation beam exiting the ATR crystal may be detected by a detector that coupled to a processing device. The processing device may determine the absorption spectrum of the CCI from the detected radiation beam. For example, FIG. 2 illustrates an example IR absorption spectrum of the CCI determined empirically through ATR-FTIR spectroscopy.

Referring back to FIG. 1, in step 130, a Stokes shift may be applied to the determined absorption spectrum using a mathematical model (“MM-130”). A Stokes shift as used herein refers to the difference in wavelength or frequency between positions of a band maxima of an absorption spectrum and an emission spectrum for the same electronic transition in a molecule. Band maximum as used herein refers to a peak absorption or emission of a band in an absorption spectrum or an emission spectrum, respectively. Hence, applying a Stokes shift to a wavelength of an absorption band may provide an approximated wavelength of the corresponding emission band. The Stokes shift applied to the absorption spectrum may be determined from absorption and emission spectra of a compound having a similar molecular structure as the identified CCI, according to an embodiment. Alternatively, the Stokes shift may be determined from molecular modeling of the identified CCI. For application of the Stokes shift, the determined Stokes shift values may be fed into MM-130 along with the determined absorption spectrum. MM-130 may perform a lateral shift of the absorption spectrum by adding the determined Stokes shift values to corresponding band maxima of the absorption spectrum.

In an embodiment, a Stokes shift may be applied to all absorption band maxima of the determined absorption spectrum. Alternatively, the Stokes shift may be applied to selected one or more primary wavelength peaks of the determined absorption spectrum. For example, peaks 202, 204, and 206 of the absorption spectrum of FIG. 2 may be selected as the primary wavelength peaks. A Stokes shift corresponding to peaks 202, 204, and 206 may be applied using MM-130. The selected one or more primary wavelength peaks may be manually input to MM-130 or may be selected by the MM-130 based on a selection criteria provided in MM-130, according to embodiments.

In step 140, an emission spectrum of the identified CCI is approximated based on the application of a Stokes shift to the determined absorption spectrum of the CCI in step 130. MM-130 outputs an approximate emission spectrum corresponding to the Stokes-shifted absorption spectrum of the CCI according to an embodiment. in another embodiment, MM-130 outputs one or more primary wavelength peaks of the identified CCI's emission spectrum corresponding to the Stokes-shifted one or more primary wavelength peaks of the identified CCI's absorption spectrum.

In step 150, a radiation signal is artificially generated based on the identified

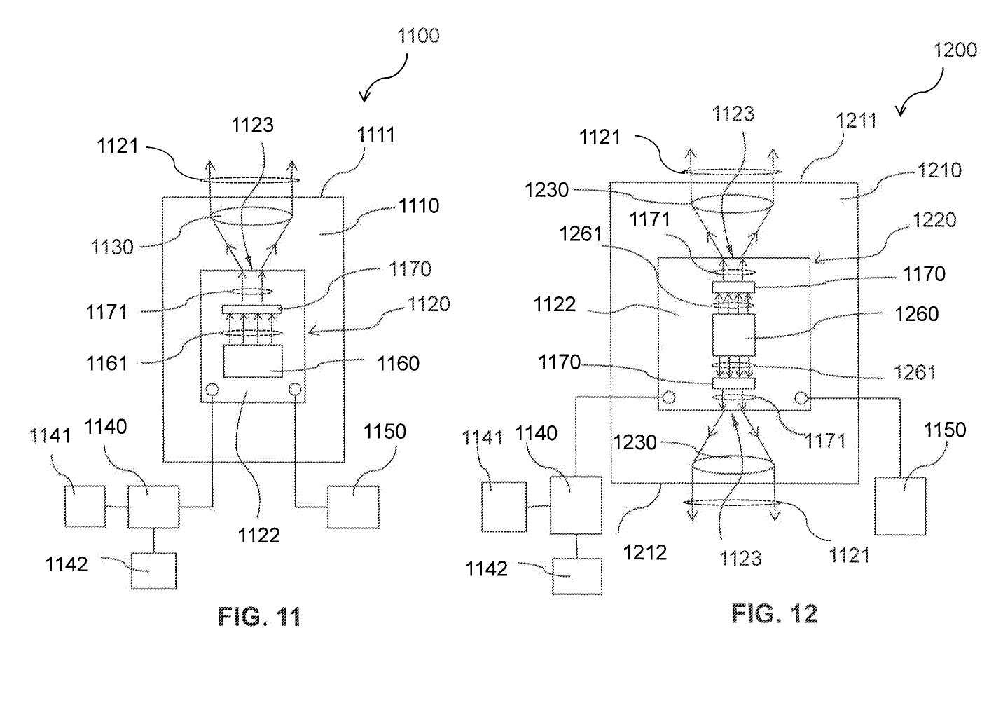

CCI's approximated emission spectrum. The artificial generation of the radiation signal may involve developing a mathematical model (“MM-150”) based on the approximated emission spectrum or approximated one or more primary wavelengths of the emission spectrum, according to embodiments. The developed MM-150 may be used to program a radiating emitter to emit signals corresponding to one or more wavelengths of the approximated emission spectrum, according to embodiments. Example embodiments of a radiating emitter and the programming of the radiating emitter are described below with reference to FIGS. 11 and 12.

It should be understood that the mathematical models MM-130 and MM-150 described above may be different mathematical models or may be parts of a mathematical model having different algorithms.

### A METHOD OF ARTIFICIALLY SIMULATING EMISSION OF A CHEMICAL COMPOUND ACCORDING TO ANOTHER EMBODIMENT

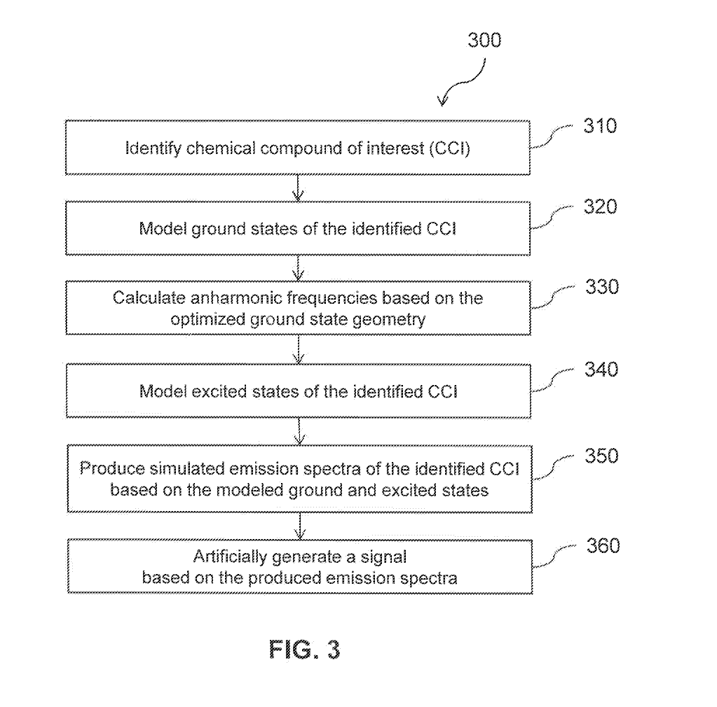

FIG. 3 illustrates a flowchart for a method of artificially simulating photonic emission of a CCI based on molecular modeling of the CCI, according to another embodiment. FIG. 3 illustrates a flowchart for a method of artificially simulating emission of a CCI according to an embodiment.

In step 310, a CCI is identified. The identifying may be based on the type of insect species targeted for inducing one or more of the behavioral responses discussed above. An example CCI is the primary sex pheromone component, Z,E-9,12-Tetradecadienyl Acetate, used to attract the male Indian Meal Moth, some other stored product moths, and some male armyworm adults.

In step 320, ground states of a molecule of the identified CCI is modeled or predicted. A molecule may have discrete energy levels. The lowest energy levels occupied by electrons of the molecule are referred as ground states of the molecule. The ground states of the identified CCI may be modeled through a molecular modeling method. Molecular modeling may concentrate on predicting the behavior of electrons in individual molecules within the CCI. The ground states may be modeled using various approaches to molecular modeling such as semi-empirical methods, molecular mechanics methods, molecular dynamics methods, “ab initio” (or “first principles”) electronic structure methods, or density functional theory (DFT) methods, according to various embodiments. DFT may be also considered as one of the “ab initio” methods. According to various embodiments, the ground states may be modeled using “ab initio” methods such as, but not limited to, Hartree-Fock methods, Post Hartree-Fock methods, or DFT methods. Hartree-Fock methods is one of the first successful methods and is used as a starting point for more elaborate Post-Hartree-Fock methods. Post-Hartree-Fock methods may include electron correlations which are only averaged in the original Hartree-Fock method. There may be multi-reference methods related to the Post-Hartree-Fock methods that include multi-configurational self-consistent field, multi-reference single and double configuration interaction, and N-electron valence state perturbation theory. These methods use more than one determinant and therefore are not strictly Post-Hartree-Fock methods. DFT methods may attempt to address both the inaccuracy and the high computational demands of Hartree-Fock and Post-Hartree-Fock methods by replacing many-body electronic wavefunction with electronic density as a basic quantity. Calculations in DFT methods may be performed using local density approximations (LDA), generalised gradient approximations (GGA), or hybrid of GGA and Hartree-Fock terms, according to various embodiments. LDA functionals contain terms related to electron density, while GGA functionals contain terms that depend upon both electron density and density gradients. The hybrid methods may provide more accurate calculations by combining GGA's electronic density functionals with Hartree-Fock's correction of self-interaction of the electrons.

Step 320 may include sub-steps 410-470, illustrated in FIGS. 4, that are involved in modeling the ground states of the identified Ca In sub-step 410, a molecular modeling method is selected. The molecular modeling method selected may be one of the above-described methods or any other conventional method appropriate for molecular modeling of a chemical compound. In sub-step 420, an algorithm for the selected molecular modeling method is selected. For example, in an embodiment, a B3LYP algorithm may be selected for using a DFT molecular model for modeling the ground states of the CCI. The B3LYP algorithm is a hybrid functional in which the exchange energy from, for example, Becke's exchange functional, is combined with the exact energy from Hartree-Fock theory. Along with the component exchange and correlation functionals, three parameters define the hybrid functional, specifying the amount of exchange energy in the hybrid functional. According to an embodiment, the selected algorithm may be executed in a Gaussian modeling software.

In sub-step 430, a basis set for carrying out calculations of the selected molecular model is selected. For example, in an embodiment, the 6-31 G* basis set may be selected. The 6-31 G* basis set defined for the atoms H through Zn is a valence double-zeta polarized basis set that adds to the 6-31 G* set six d-type Cartesian-Gaussian polarization functions on each of the atoms Li through Ca and ten f-type Cartesian Gaussian polarization functions on each of the atoms Sc through Zn. A basis set may be referred as a set of functions (called basis functions) which are combined in linear combinations (generally as part of a quantum chemical calculation) to create molecular orbitals,. Generally, accuracy of the results may depend on the degree of electron correlation and the size of the basis set used. The processing time required for some parts of an “ab initio” or DFT calculation may be dependent on the number of basis functions in an embodiment. Hence, cost of calculations may increase with increase in the basis set size is and the amount of electron correlations. In an embodiment, the selected basis set is executed in the Gaussian modeling software package.

In sub-step 440, an optimization of ground state geometry for the identified CCI is performed. Geometry optimization refers to a method of taking rough geometric approximations and making them as exact as possible. The starting molecular geometry of the CCI selected for the optimization may be a representation of the atomic make-up of the CCI provided by the manufacturer or a manually-constructed representation of the atomic make-up of the CCI using a graphic interface like GaussView, according to embodiments. Geometry Optimization may require many cycles in order to move the atoms around in a way so as to minimize the energy. This minimization may be performed by computing the forces on each atom, and performing an iterative procedure in which the atoms are moved slightly in steps until the energy gradient is minimized. The energy gradient may be referred as the derivative of energy with respect to motion of all the atoms. Optimized ground state geometry may be achieved when the energy gradient is zero, indicating a minimum in the Potential Energy Surfaces (PES), otherwise the selected molecular geometry may be modified and the geometry optimization cycle may be repeated. In an embodiment, the geometry optimization is executed in the Gaussian modeling software package.

The computations in the geometry optimization of sub-step 440 may assume an idealized view of nuclear position in the molecular structure of the CCI and ignore the vibrations present in the molecule (Born-Oppenheimer Approximation). In reality, the nuclei in molecules are constantly in motion, and in equilibrium states, these vibrations are regular and predictable, and molecules can be identified by their characteristic spectra. Hence, to account for the presence of these vibrations in the molecular structure of the CCI, harmonic vibrational frequencies may he calculated in sub-step 450 based on the optimized geometry of the CCI's ground states (sub-step 440). Frequency calculations depend on the second derivative of the electron energy with respect to the nuclear positions. The frequency calculations may output eigenvalues (frequencies) and eigenvectors (normal modes). Imaginary frequencies may be represented by negative frequencies in the output. In an embodiment, the frequency calculations may be executed in the Gaussian modeling software package.

IA sub-step 460, the results from the frequency calculations are graphically represented to provide a simulated IR absorption spectrum of the identified CCI. The vibrational intensities may be calculated from the transition moment integral using various harmonic wave functions. There may be a systematic error in the calculated harmonic vibrational frequency when compared to experimental fundamental vibrational frequency. This may be attributed in part to inaccurate description of electron-electron interaction and the neglect of anharmonicity in the vibration intensity calculations. Therefore scaling factors may be applied to compensate for known deviations between model and experimental information and/or calculations for anharmonicity may be applied in the model.

In sub-step 470, a fit of the selected molecular model (sub-step 410) for modeling the ground states of the identified CCI is evaluated. In an embodiment, the evaluation is performed by fitting the simulated IR absorption spectrum to an empirically determined IR absorption spectrum of the identified CCI. In another embodiment, the evaluation may be performed by checking the number of imaginary frequencies in the frequency calculations (sub-step 460). Presence of imaginary frequencies may indicate an unstable model of the ground states. Sub-steps 410-470 may be repeated with a different selected basis set, algorithm, molecular modeling method, or any combination thereof until a best fit is obtained between the projected and empirical IR absorption spectra or a stable model is determined.

Referring back to FIG. 3, following the modeling of the ground states in step 320, anharmonic frequencies in the molecular structure of the CCI are calculated in step 330. The anharmonic frequencies may be calculated based on the optimized ground state geometry obtained in sub-step 440. While step 330 is shown as being performed after step 320, the anharmonic frequency calculations of step 330 may instead be performed subsequent to step 340 described below, and/or as part of step 320 and/or part of step 340 according to an embodiment. In alternate embodiments, step 330 may be an optional step.

In step 340, excited states of a molecule of the identified CCI are modeled or predicted. An excited state of a molecule may refer to any quantum state of the molecule that has a higher energy than the ground state. The excited states may be modeled using various methods of molecular modeling such as Configuration Interaction with Single Excitations (CIS) methods, Time-Dependent Density Functional Theory (TD-DFT) methods, Post Hartree-Fock methods, or Multi-Reference methods, according to various embodiments. Step 340 may include sub-steps 510-570, as illustrated in FIG. 5, that are involved in modeling the excited states of the identified CCI. Additionally or alternatively, step 340 may include sub-steps 610-670, as illustrated in FIG. 6 for modeling the excited states of the identified CCI.

In sub-step 510, a molecular modeling method is selected. The molecular modeling method selected may be one of the above-described methods for molecular modeling of the excited states. Subsequent sub-steps 520-530 are similar to the sub-steps 420-430 of FIGS. 4 as described above. In sub-step 540 a single point calculation of the excited states is performed based on the ground state optimization of sub-step 440 described above.

In sub-step 550, frequency calculations may be performed as part of the Franck-Condon-Herzberg-Teller method. In sub-step 560, the results from the single point calculation and any frequency calculations are graphically represented to provide a simulated UV-Vis absorption spectra of the identified CCI.

In sub-step 570, a fit of the selected molecular model for modeling the excited states of the identified CCI is evaluated. Sub-steps 510-570 may be repeated with a different selected basis set, algorithm, molecular modeling method, or any combination thereof until a best fit is obtained between the projected and empirical absorption spectra or a stable model is determined.

In sub-step 610, a molecular modeling method is selected. The molecular modeling method selected may be one of the above-described methods for molecular modeling of the excited states. Subsequent sub-steps 620-630 are similar to the sub-steps 420-430 of FIGS. 4 as described above. In sub-step 640, an optimization of excited state geometry is performed. Sub-step 640 is similar to sub-step 440 of FIGS. 4 as described above.

In sub-step 650, frequency calculations are performed similarly to sub-step 450 of FIGS. 4 as described above. In sub-step 660, the results from the frequency calculations are graphically represented to provide a simulated excited state IR vibrational spectrum of the identified CCI. In an embodiment, another frequency calculation known as the Franck-Condon-Herzberg-Teller method may determine a further Stokes shift of the simulated UV-Vis emission spectrum of the CCI. A simulated UV-Vis emission spectrum may be extracted from the optimized excited state geometry.

In sub-step 670, the evaluation is performed by checking the number of imaginary frequencies in the frequency calculations (sub-step 660). Presence of imaginary frequencies may indicate an unstable model of the excited states. Sub-steps 610-670 may be repeated with a different selected basis set, algorithm, molecular modeling method, or any combination thereof until a best fit is obtained between the projected and empirical absorption spectra or a stable model is determined.

Referring back to FIG. 3, following the modeling of the excited states (step 340), in step 350, a simulated IR emission spectrum is produced based on the frequency calculations of the selected molecular model for the modeled excited and ground states.

In step 360, a radiation signal is artificially generated based on the identified CCI's emission spectrum. The artificial generation of the radiation signal may involve developing a mathematical model (“MM-360”) based on the modeled emission spectrum. The developed MM-360 may be used to program a radiating emitter to emit signals corresponding to wavelengths of the modeled emission spectrum, according to an embodiment. In another embodiment, MM-360 is used to select one or more primary wavelengths of the modeled emission spectrum based on selection criteria provided in MM-360 and program the radiating emitter to emit signals corresponding to these selected one or more primary wavelengths. Example embodiments of a radiating emitter and the programming of the radiating emitter are described below with reference to FIGS. 11 and 12.

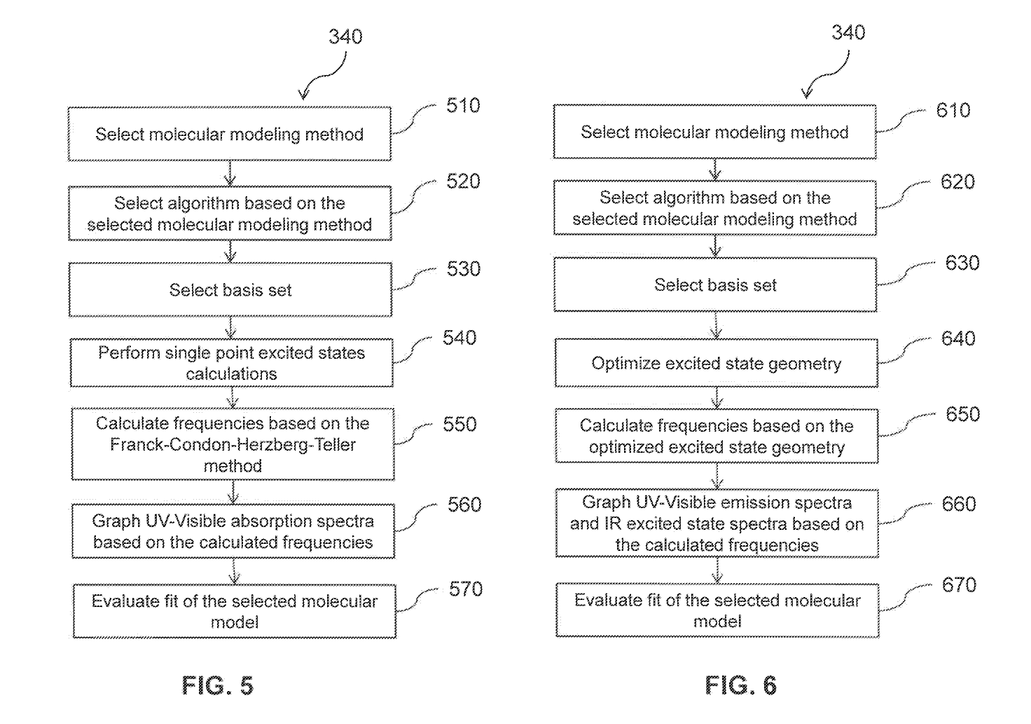

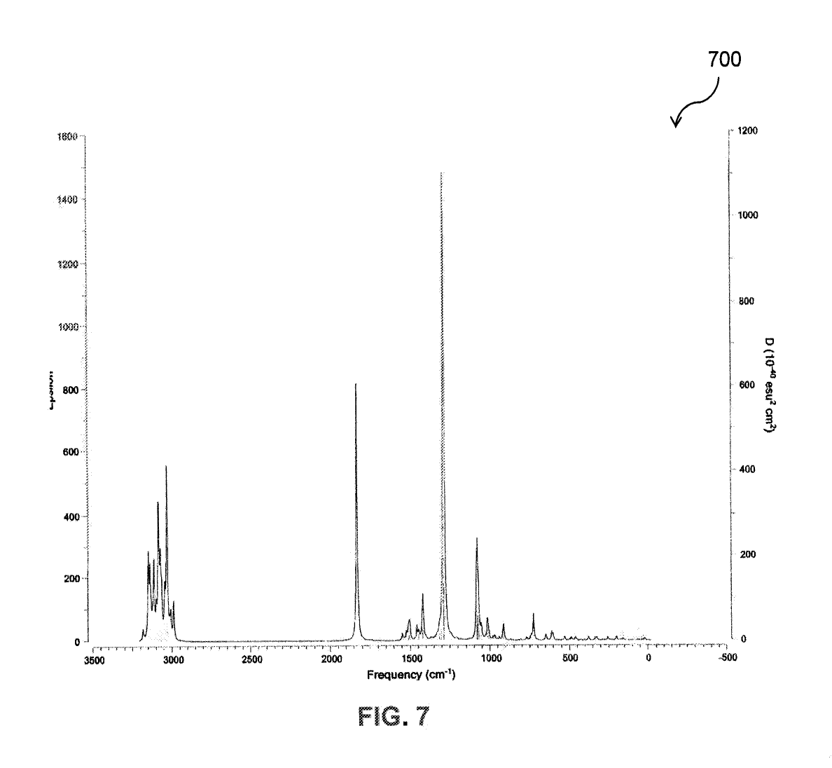

FIGS. 7 and 8 illustrate plots of an example simulated IR absorption spectrum and an example simulated excited state IR vibrational spectrum of a pheromone molecule, respectively, determined using a molecular modeling method similar to that described above with reference to FIGS. 3, 4, 5, and 6.

### A METHOD OF ARTIFICIALLY SIMULATING EMISSION OF A CHEMICAL COMPOUND ACCORDING TO ANOTHER EMBODIMENT

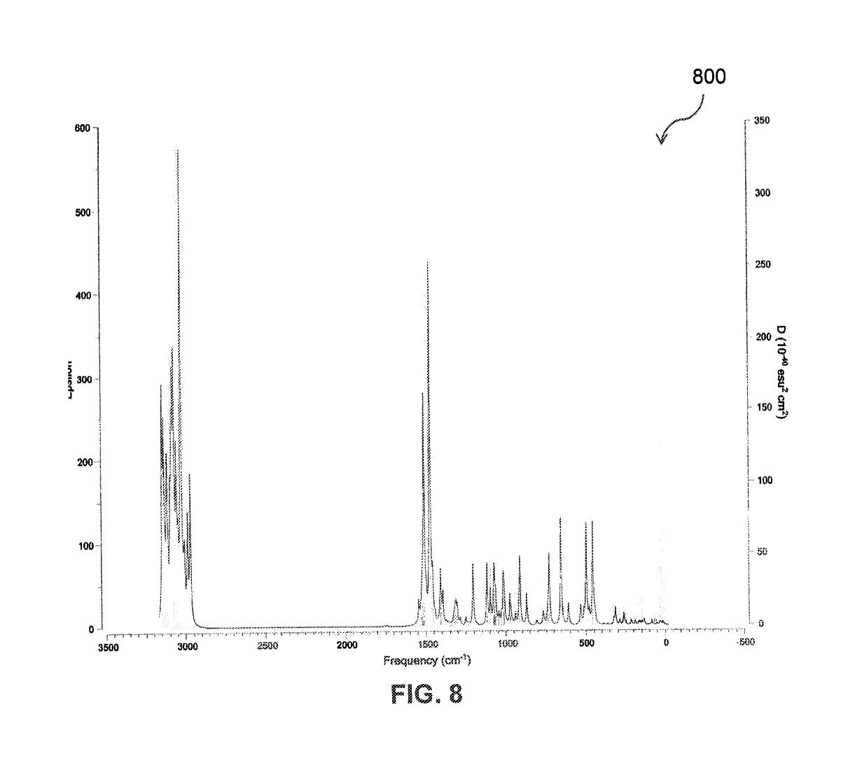

FIG. 9 illustrates a flowchart for a method of artificially simulating photonic emission of a CCI according to another embodiment.

In step 910, a CCI is identified. The identifying may be based on the type of insect species targeted for inducing one or more of the behavioral responses discussed above. An example is, the sex pheromone known to attract male Indian Meal Moths.

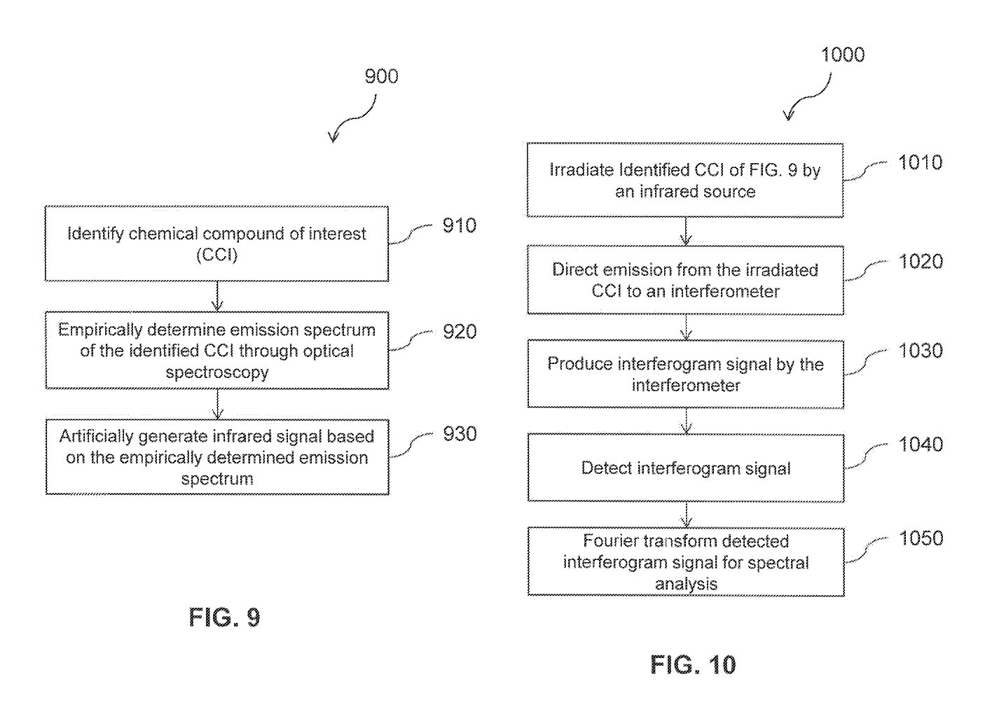

In step 920, an emission spectrum of the identified CCI is determined empirically through optical spectroscopy such as, but not limited to, Fourier transform infrared (FT-IR) spectroscopy. Example steps involved in determining IR emission spectrum of the identified CCI through FT-IR spectroscopy are illustrated in FIG. 10. In step 1010, the identified CCI is irradiated by a non-coherent and continuous radiation beam from a thermal IR source. Subsequently, emission from the irradiated CCI is directed to an interferometer in step 1020, such as a Michelson interferometer. In step 1030, the interferometer produces an interferogram signal corresponding to the emission wavelengths received from the irradiated CCI. The produced interferogram signal has the unique property that every data point of the signal has information about every infrared frequency of the emission received from the irradiated CCI. In following step 1040, the interferogram signal is detected by a detector coupled to a processing device. The processing device may Fourier transform the detected interferogram for spectral analysis of the emission from the irradiated CCI in step 1050.

Referring back to FIG. 9, following step 920, an IR radiation signal is generated artificially in step 930 based on the empirically-determined emission spectrum. The artificial generation of the IR radiation signal may involve developing a mathematical model (“MM-930”) based on the empirically-determined emission spectrum. The developed MM-930 may be used to program an radiating emitter to emit IR signals corresponding to wavelengths of the empirically-determined emission spectrum, according to an embodiment. In another embodiment, MM-930 is used to select one or more primary wavelengths of the empirically-determined emission spectrum based on a selection criteria provided in MM-930 and program the radiating emitter to emit IR signals corresponding to these selected one or more primary wavelengths. Example embodiments of a radiating emitter and the programming of the radiating emitter are described below with reference to FIGS. 11 and 12.

### APPARATUS FOR AFFECTING BEHAVIOR OF AN INSECT SPECIES ACCORDING TO AN EMBODIMENT

FIGS. 11 illustrates a schematic diagram of an apparatus 1100 for inducing behavioral responses in an insect species, according to an embodiment. Apparatus 1100 includes a housing 1110, a radiating emitter 1120, a directing apparatus 1130, a processing device 1140, a power source 1150, weather sensors 1141, and environmental sensors 1142.

In an embodiment, housing 1110 is configured to hold radiating emitter 1120 and directing apparatus 1130. While power source 1150 and processing device 1140 are shown in FIGS. 11 to be placed outside of housing 1110, they may be placed within housing 1110 in alternate embodiments. Housing 1110 is not restricted to having a straight-sided shape as illustrated schematically in FIGS. 11; rather it may be configured to be any type of geometric shape, such as but not limited to cuboid, cylindrical, spherical, or elliptical. As shown in FIG. 11, a side 1111 of housing 1110 may be transmissive to directed radiation 1121 emitted from radiating emitter 1120. Optically transmissive side 1111 may include a UV, Vis, or IR transmissive window (not shown) strategically positioned to allow emission of directed radiation 1121 from radiating emitter 1120, in an embodiment. Alternatively, optically transmissive side 1111 may include a window (not shown) that forms the side 1111 of housing 1110. In an embodiment, housing 1110 is configured to be weather resistant and capable of being mounted or portably deployed in agricultural and stored grain environments.

According to an embodiment, radiating emitter 1120 includes a casing 1122, a radiating source 1160, and an optical filter system 1170. Casing 1122 may be configured to hold radiating source 1160 and optical filter system 1170. Radiating source 1160 may be configured to emit radiation 1161. Radiating source 1160 may include one or more devices such as, but not limited to, a blackbody radiator, one or more light emitting diodes, or one or more lasers. Optical filter system 1170 may include a plurality of optical filters with each optical filter configured to selectively allow transmission of a different wavelength or a different set of wavelengths of UV, Vis, or IR radiation 1161. The optically-filtered radiation 1171 may be emitted from radiating emitter 1120 through an aperture 1123 included in casing 1122. Alternatively, radiating emitter 1120 may include only radiating source 1160.

In an embodiment, directing apparatus 1130 is configured to direct filtered radiation 1171 exiting casing 1122 of radiating emitter 1120 in a desired direction. The position of directing apparatus 1130 may be controlled by a main controller (not shown) in an embodiment.

In an embodiment, processing device 1140 is coupled to radiating emitter 1120. Processing device 1140 may be configured to program radiating emitter 1120 to emit radiation 1171 having one or more wavelengths of an emission spectrum of one or more CCIs identified for inducing behavioral responses in targeted insect species. Programming radiating emitter 1120 may involve executing a mathematical model or internally storing or receiving via wired or wireless telemetry the results of an externally executed mathematical model such as, but not limited to, MM-150, MM-360, or MM-930 for one or more CCIs. These mathematical models as described above with reference to FIGS. 1, 3, 4, 5, and 6 may be developed based on the emission spectrum of a CCI determined empirically, semi-empirically, or by modeling. Based on the execution of one or more of these mathematical models, processing device 1140 may enable operation of radiating source 1160 and select optical filters in optical filter system 1170 to produce radiation 1171. The spectrum of produced radiation 1171 may mimic the empirical or modeled emission spectrum or primary wavelengths of the CCIs identified for inducing behavioral responses in targeted insect species. The spectrum of produced radiation 1171 may also include additional wavelengths that were not empirically determined or modeled. The spectrum of produced radiation 1171 may also include only a subset of the total number of wavelengths empirically determined or modeled.

In an embodiment, power source 1150 is coupled to radiating emitter 1120. Power source 1150 may be a battery or any type of power supply, including but not limited to regulated or unregulated, solar cells, and power supplies that can be externally controlled or modulated in order to produce varying voltages, currents, and waveforms. Power source 1150 may be configured to control an intensity of radiation 1171 emitted from radiating emitter 1120. The intensity of radiation 1171 may be controlled by regulating the power of radiating source 1160. Increasing the intensity of radiation 1171 may increase the volumetric area in which insect species' behavior may be affected. The greater the intensity of radiation 1171, the more likely the insect species will be able to detect and react to the radiation. Power control of radiation 1171 may also provide ecological benefits over pesticides used for pest population control. While pesticides deployed in an area tend to wipe out the targeted insect species in that area, controlling the radiated output may limit reproduction of the targeted insect species instead of annihilating them, thus helping to minimize ecological destruction.

In an embodiment, processing device 1140 is configured to cause power source 1150 to produce various types of emissions from radiating source 1160. Examples of various types of emissions include, but are not limited to, continuous wave, pulsed, pulse-width-modulation, amplitude modulation, frequency modulation, or any combination thereof Processing device 1140 may be further configured for daytime, nighttime, or any type of time-of-day programming control of power source 1150 and radiating source 1160, according to an embodiment. Weather sensors 1141 and environmental sensors 1142 may be connected to processing device 1140 to allow processing device 1140 independent algorithmic control over power source 1150 and radiating source 1160 as a function of environmental or weather conditions. For instance, processing device 1140 may disable power source 1150 and radiating emitter 1160 when it is raining, when the temperature falls below or exceeds predetermined thresholds, or when it is daylight or nighttime. Processing device 1140 may connect directly or indirectly to keypad inputs, display screens, various I/O, and other ancillary devices (not shown) well known to those skilled in the art.

In an embodiment, radiating source 1160 may be directly connected to power source 1150. Radiating source 1160 may be a single LED or an arrangement of LEDs emitting radiation at one or more wavelengths of an emission spectrum of one or more CCIs identified for inducing behavioral responses in a targeted insect species.

It should be noted that, for the sake of simplicity, apparatus 1100 is shown in FIG. 11 as including only one arrangement of radiating emitting device 1160, optical filter system 1170, and directing apparatus 1130 for emitting radiation 1121 through only one side 1111 of housing 1110. However, as would be understood by a person skilled in the art based on the description herein, apparatus 1100 may be configured to emit, or may include any number of such arrangements configured to emit, radiation through other optically transmissive sides of housing 1110. In an embodiment, apparatus 1100 may simply include power source 1150 and radiating source 1160 that is configured to emit radiation 1161.

### APPARATUS FOR AFFECTING BEHAVIOR OF INSECT SPECIES ACCORDING TO ANOTHER EMBODIMENT

FIG. 12 illustrates a schematic diagram of an apparatus 1200 for inducing behavioral responses in insect species according to another embodiment. Apparatus 1200 includes a housing 1210, a radiating emitter 1220, a directing system 1230, a processing device 1140, a power source 1150, weather sensors 1141, and environmental sensors 1142.

In an embodiment, housing 1210 is configured to hold radiating emitter 1120 and directing system 1230. Housing 1210 may be transmissive to directed radiations 1121 emitted from radiating emitter 1120 according to an example embodiment. As shown in FIG. 12, sides 1211 and 1212 of housing 1210 may be configured to be optically transmissive to radiations 1121 emitted in multiple directions from radiating emitter 1220, according to an example embodiment. Optically transmissive sides 1211 and 1212 may each include a UV, Vis, or IR transmissive window (not shown) strategically positioned to allow emission of directed radiation 1121. Alternatively, sides 1211 and 1212 may each include a transmissive window (not shown) that forms sides 1211 and 1212 of housing 1210. Alternatively, all sides of housing 1200 may be transmissive to radiations emitted from radiating emitter 1220.

According to an embodiment, radiating emitter 1220 includes a casing 1122, a radiating source 1260, and an optical filter system 1170. Casing 1122 may be configured to hold radiating source 1260 and optical filter system 1170. Radiating source 1260 may be configured to emit radiation 1261 in a plurality of directions. Radiating source 1260 may include one or more devices such as, but not limited to, a blackbody radiator or light emitting diodes. Optical filter system 1170 may include a plurality of optical filters, with each optical filter configured to selectively allow transmission of a different wavelength or a different set of wavelengths of radiation 1161. The optically-filtered radiation 1171 may be emitted from radiating emitter 1120 through apertures 1123 included in casing 1122 (FIG. 12).

In an embodiment, directing system 1230 includes a plurality of directing apparatuses similar to directing apparatus 1130. The position of each directing apparatus 1130 may be controlled by a main controller or individual controller (not shown) according to various embodiments.

In an embodiment, processing device 1140 is coupled to radiating emitter 1220. Processing device 1140 may be configured to program radiating emitter 1220 in a manner similar to that described for radiating emitter 1120 with reference to FIG. 11. Power source 1150 may be coupled to radiating emitter 1220 and may be configured to control intensities of radiation 1171 emitted from radiating emitter 1220. Intensity of radiation 1171 may be controlled by regulating the power of radiating source 1260. Power source 1150 may be a battery or any type of power supply, including but not limited to regulated or unregulated, solar cells, and power supplies that can be externally controlled or modulated in order to produce varying voltages, currents, and waveforms.

In an embodiment, processing device 1140 is configured to cause power source 1150 to produce various types of emissions from radiating source 1260 in a manner similar to that described for radiating source 1160 with reference to FIG. 11. Processing device 1140 may be further configured for daytime, nighttime, or any type of time-of-day programming control of power source 1150 and radiating source 1260, according to an embodiment. Weather sensors 1141 and environmental sensors 1142 may be connected to processing device 1140 to allow processing device 1140 independent algorithmic control over power source 1150 and radiating source 1260 as a function of environmental or weather conditions. For instance, processing device 1140 may disable power source 1150 and radiating source 1260 when it is raining, when the temperature falls below or exceeds predetermined thresholds, or when it is daylight or nighttime. Processing device 1140 may connect directly or indirectly to keypad inputs, display screens, various I/O, and other ancillary devices (not shown) well known to those skilled in the art.

### AN EXAMPLE COMPUTER SYSTEM

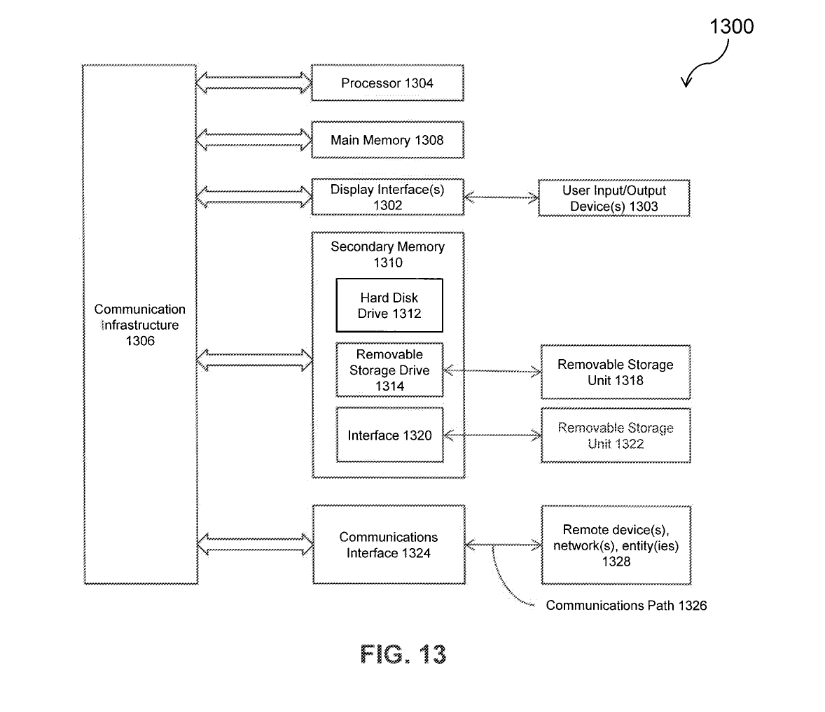

Various aspects of the present invention may be implemented in software, firmware, hardware, or a combination thereof FIG. 13 is an illustration of an example computer system 1300 in which embodiments of the present invention, or portions thereof, can be implemented as computer-readable code. For example, the methods illustrated by flowcharts 100, 300, 400, 500, 600, 900, and 1000 of FIGS. 1, 3-6 and 9-10, respectively, can be implemented in system 1300. Various embodiments of the present invention are described in terms of this example computer system 1300. After reading this description, it will become apparent to a person skilled in the relevant art how to implement embodiments of the present invention using other computer systems and/or computer architectures.

It should be noted that the simulation, synthesis and/or manufacture of various embodiments of this invention may be accomplished, in part, through the use of computer readable code, including general programming languages (such as C or C++), hardware description languages (HDL) such as, for example, Verilog HDL, VHDL, Altera HDL (AHDL), or other available programming and/or schematic capture tools (such as circuit capture tools). This computer readable code can be disposed in any known computer-usable medium including a semiconductor, magnetic disk, optical disk (such as CD-ROM, DVD-ROM). As such, the code can be transmitted over communication networks including the Internet. It is understood that the functions accomplished and/or structure provided by the systems and techniques described above can be represented in a memory.

Computer system 1300 includes one or more processors, such as processor 1304. Processor 1304 is connected to a communication infrastructure 1306 (e.g., a bus or network).

Computer system 1300 also includes a main memory 1308, such as random access memory (RAM), and may also include a secondary memory 1310. Secondary memory 1310 can include, for example, a hard disk drive 1312, a removable storage drive 1314, and/or a memory stick. Removable storage drive 1314 can include a floppy disk drive, a magnetic tape drive, an optical disk drive, a flash memory, or the like. The removable storage drive 1314 reads from and/or writes to a removable storage unit 1318 in a well-known manner. Removable storage unit 1318 can include a floppy disk, magnetic tape, optical disk, flash drive, etc. which is read by and written to by removable storage drive 1314. As will be appreciated by persons skilled in the relevant art, removable storage unit 1318 includes a computer-readable storage medium having stored therein computer software and/or data.

Computer system 1300 (optionally) includes a display interface 1302 (which can include input and output devices 1303 such as keyboards, mice, etc.) that forwards graphics, text, and other data from communication infrastructure 1306 (or from a frame buffer not shown) for display on display unit 1330.

In alternative implementations, secondary memory 1310 can include other similar devices for allowing computer programs or other instructions to be loaded into computer system 1300. Such devices can include, for example, a removable storage unit 1322 and an interface 1320. Examples of such devices include a program cartridge and cartridge interface (such as those found in video game devices), a removable memory chip (e.g., EPROM or PROM) and associated socket, and other removable storage units 1322 and interfaces 1320 which allow software and data to be transferred from the removable storage unit 1322 to computer system 1300.

Computer system 1300 can also include a communications interface 1324. Communications interface 1324 allows software and data to be transferred between computer system 1300 and external devices. Communications interface 1324 can include a modem, a network interface (such as an Ethernet card), a communications port, a PCMCIA slot and card, or the like. Software and data transferred via communications interface 1324 are in the form of signals which may be electronic, electromagnetic, optical, or other signals capable of being received by communications interface 1324. These signals are provided to communications interface 1324 via a communications path 1326. Communications path 1326 carries signals and can be implemented using wire or cable, fiber optics, a phone line, a cellular phone link, a RF link or other communications channels.

In this document, the terms “computer program storage medium” and “computer-readable storage medium” are used to generally refer to non-transitory media such as removable storage unit 1318, removable storage unit 1322, and a hard disk installed in hard disk drive 1312. Computer program storage medium and computer-readable storage medium can also refer to memories, such as main memory 1308 and secondary memory 1310, which can be memory semiconductors (e.g., DRAMs, etc.). These computer program products provide software to computer system 1300.

Computer programs (also called computer control logic) are stored in main memory 1308 and/or secondary memory 1310. Computer programs may also be received via communications interface 1324. Such computer programs, when executed, enable computer system 1300 to implement embodiments of the present invention as discussed herein. In particular, the computer programs, when executed, enable processor 1304 to implement processes of embodiments of the present invention, such as the steps in the methods illustrated by flowchart 100, 300, 400, 500, 600, 900, and 1000 of FIGS. 1, 3-6 and 9-10, respectively, can be implemented in system 1300, discussed above. Where embodiments of the present invention are implemented using software, the software can be stored in a computer program product and loaded into computer system 1300 using removable storage drive 1314, interface 1320, hard drive 1312, or communications interface 1324.

Embodiments of the present invention are also directed to computer program products including software stored on any computer-readable storage medium. Such software, when executed in one or more data processing device, causes a data processing device(s) to operate as described herein. Embodiments of the present invention employ any computer-readable medium, known now or in the future. Examples of computer-readable storage mediums include, but are not limited to, non-transitory primary storage devices (e.g., any type of random access memory), and non-transitory secondary storage devices (e.g., hard drives, floppy disks, CD ROMS, ZIP disks, tapes, magnetic storage devices, optical storage devices, MEMS, nanotechnological storage devices, etc.). Embodiments of the present invention may alternatively employ communication mediums (e.g., wired and wireless communications networks, local area networks, wide area networks, intranets, etc.).

While various embodiments of the present invention have been described above, it should be understood that they have been presented by way of example, and not limitation. It will be apparent to persons skilled in the relevant art(s) that various changes in form and detail can be made therein without departing from the spirit and scope of the invention. Thus, the present invention should not be limited by any of the above described exemplary embodiments, but should be defined only in accordance with the following claims and their equivalents.

What is claimed is:

1. An apparatus for causing a behavioral response in an insect species, comprising:
a housing;
a radiating emitter disposed within the housing, the radiating emitter configured to emit radiation at one or more wavelengths simulating an emission spectrum of a chemical compound of interest, the chemical compound of interest causing a behavioral response in the insect species;
a directing apparatus configured to direct the emitted radiation; and
a power source coupled to the radiating emitter and configured to control an intensity of the emitted radiation.

2. The apparatus of claim 1, wherein the emitted radiation includes infrared radiation.

3. The apparatus of claim 1, wherein the emitted radiation includes visible radiation.

4. The apparatus of claim 1, wherein the emitted radiation includes ultraviolet radiation.

5. The apparatus of claim 1, wherein the radiating emitter comprises:
at least one optical filter; and
a blackbody radiator configured to transmit radiation at the one or more wavelengths simulating an emission spectrum through the at least one optical filter.

6. The apparatus of claim 1, wherein the radiating emitter comprises:
at least one optical filter; and
a plurality of light emitting diodes configured to transmit radiation at the one or more wavelengths simulating an emission spectrum through the at least one optical filter.

7. The apparatus of claim 1, wherein the radiating emitter comprises:
at least one optical filter; and
one or more lasers configured to transmit radiation at the one or more wavelengths simulating an emission spectrum through the at least one optical filter.

8. The apparatus of claim 1, wherein the radiating emitter is also programmed to emit radiation at one or more wavelengths simulating an emission spectrum of a different chemical compound of interest, the different chemical compound of interest causing a behavioral response in a different insect species.

9. The apparatus of claim 1, wherein the radiating emitter is also programmed to emit radiation at one or more wavelengths simulating an emission spectrum of a different chemical compound of interest, the different chemical compound of interest causing a different behavioral response in the insect species.

10. The apparatus of claim 1, wherein a power of the radiating emitter is variable.

11. The apparatus of claim 1, wherein a power of the radiating emitter is programmable.

12. The apparatus of claim 1, wherein the radiating emitter is configured to emit radiation between 300 nm and 30 μm.

13. The apparatus of claim 1, wherein the one or more wavelengths simulating an emission spectrum of a chemical compound of interest is determined empirically.

14. The apparatus of claim 1, wherein the one or more wavelengths simulating an emission spectrum of a chemical compound of interest is determined by applying a Stokes shift to a radiation absorption spectrum of the chemical compound of interest.

15. The apparatus of claim 1, wherein the chemical compound of interest is a semiochemical.

16. The apparatus of claim 15, wherein the semiochemical is at least one of a pheromone, a kairomone, an allomone, or a synomone.

17. The apparatus of claim 1, wherein the chemical compound of interest is an odorant.

18. The apparatus of claim 1, wherein the power source is configured to control the production of a type of emission from the radiating emitter.

19. The apparatus of claim 18, wherein the type of emission produced from the radiating emitter is a continuous wave emission, pulsed emission, pulse-width-modulated emission, amplitude modulated emission, or frequency modulated emission.

20. The apparatus of claim 1, further comprising a processing device configured for time-of-day programming of the radiating emitter and the power source.

21. The apparatus of claim 20, further comprising a weather sensor coupled to the processing device, the weather sensor being configured to allow the processing device algorithm control over the radiating emitter and the power source as a function of a weather condition.

22. The apparatus of claim 20, further comprising an environmental sensor coupled to the processing device, the environmental sensor being configured to allow the processing device algorithm control over the radiating emitter and the power source as a function of an environmental condition.

23. An apparatus for causing a behavioral response in an insect species, comprising:
a radiating emitter configured to emit radiation at one or more wavelengths simulating an emission spectrum of a chemical compound of interest, the chemical compound of interest causing a behavioral response in the insect species; and
a power source coupled directly to the radiating emitter.

24. The apparatus of claim 23, wherein the chemical compound of interest is a semiochemical.

25. The apparatus of claim 24, wherein the semiochemical is at least one of a pheromone, a kairomone, an allomone, or a synomone.

26. The apparatus of claim 23, wherein the chemical compound of interest is an odorant.

27. A method of artificially simulating an emission of a chemical compound of interest, comprising:
identifying a chemical compound of interest, the chemical compound of interest causing a behavioral response in an insect species;
determining a radiation absorption spectrum of the chemical compound of interest, the absorption spectrum comprising at least one absorption wavelength value;
applying a Stokes shift to the at least one absorption wavelength value of the absorption spectrum;
approximating an emission spectrum of the chemical compound of interest based on the applying, the emission spectrum comprising at least one emission wavelength value.

28. The method of claim 27, further comprising:
artificially generating a radiation signal based on the approximated emission spectrum, wherein the radiation signal causes a behavioral response in the insect species.

29. The method of claim 28, wherein the artificially generating comprises:
developing a mathematical model based on the approximated emission spectrum; and
programming a radiating emitter to emit a radiation signal corresponding to the mathematical model.

30. The method of claim 27, wherein:
determining comprises determining values of primary wavelength peaks in the radiation absorption spectrum;
applying comprises applying a Stokes shift to the values of the primary wavelength peaks; and
approximating comprises approximating primary wavelength peaks of an emission spectrum based on the applied Stokes shift.

31. The method of claim 30, further comprising:
artificially generating a radiation signal having one or more wavelengths corresponding to the approximated primary wavelength peaks of the emission spectrum of the chemical compound of interest, wherein the radiation signal causes a behavioral response in the insect species.

32. The method of claim 27, further comprising:
programming a radiating emitter to emit radiation at one or more wavelengths of the approximated emission spectrum.

33. The method of claim 27, wherein the emission spectrum of the chemical compound of interest includes wavelengths between 300 nm and 30 μm.

34. The method of claim 27, wherein the chemical compound of interest is a semiochemical.

35. The method of claim 34, wherein the semiochemical is at least one of a pheromone, a kairomone, an allomone, or a synomone.

36. The method of claim 27, wherein the chemical compound of interest is an odorant.

37. A method of artificially simulating an emission of a chemical compound of interest, comprising:
identifying a chemical compound of interest, the chemical compound of interest causing a behavioral response in an insect species;
modeling one or more ground states of the chemical compound of interest;
modeling one or more excited states of the chemical compound of interest based on the modeled one or more ground states; and
producing an emission spectrum of the chemical compound of interest based on a frequency calculation of the modeled one or more excited states, the emission spectrum comprising at least one emission wavelength value.

38. The method of claim 37, wherein modeling one or more ground states comprises:
estimating a ground state geometry for the chemical compound of interest based on a selected modeling method, modeling algorithm, and basis set;
performing a frequency calculation based on the estimated ground state geometry; and
producing an absorption spectrum of the chemical compound of interest based on the frequency calculation.

39. The method of claim 38, wherein modeling one or more ground states further comprises:
evaluating a fit of the selected modeling method; and
estimating the ground state geometry based on a different modeling method and modeling algorithm when the fit does not meet a predefined quality criterion.

40. The method of claim 39, wherein the predefined quality criterion includes at least one of stability and empirical data.

41. The method of claim 38, wherein the selected modeling method is at least one of a semi-empirical method, a molecular mechanics method, a molecular dynamics method, an ab initio electronic structure method, or a density functional theory method.

42. The method of claim 37, wherein modeling one or more excited states comprises:
estimating an excited state geometry for the chemical compound of interest based on a selected modeling method, modeling algorithm, and basis set; and
performing the frequency calculation based on the estimated excited state geometry.

43. The method of claim 42, wherein modeling one or more excited states further comprises:
evaluating a fit of the selected modeling method; and
estimating the excited state geometry based on a different modeling method and modeling algorithm when the fit does not meet a predefined quality criterion.

44. The method of claim 43, wherein the predefined quality criterion includes at least one of stability and empirical data.

45. The method of claim 42, wherein the selected modeling method is at least one of a semi-empirical method, a molecular mechanics method, a molecular dynamics method, an ab initio electronic structure method, or a time-dependent density-functional theory method.

46. The method of claim 37, further comprising:
artificially generating a radiation signal based on the simulated emission spectrum, wherein the radiation signal causes a behavioral response in the insect species.

47. The method of claim 37, wherein the chemical compound of interest is a semiochemical.

48. The method of claim 47, wherein the semiochemical is at least one of a pheromone, a kairomone, an allomone, or a synomone.

49. The method of claim 37, wherein the chemical compound of interest is an odorant.

50. A method of artificially simulating an emission of a chemical compound of interest, comprising:
identifying a chemical compound of interest, the chemical compound of interest causing a behavioral response in an insect species; and
empirically determining an emission spectrum of the chemical compound of interest through Fourier transform infrared (FTIR) spectroscopy.

51. The method of claim 50, further comprising:
selecting one or more peak wavelengths based on the empirically determined emission spectrum; and
programming a radiating emitter to emit radiation at one or more wavelengths of the empirically determined emission spectrum.

52. The method of claim 50, further comprising:
artificially generating a radiation signal based on the empirically determined emission spectrum, wherein the radiation signal causes a behavioral response in the insect species.

53. The method of claim 50, further comprising:
artificially generating a radiation signal having one or more wavelengths corresponding to primary wavelength peaks of the empirically determined emission spectrum of the chemical compound of interest, wherein the radiation signal causes a behavioral response in the insect species.

54. The method of claim 50, wherein the emission spectrum of the chemical compound of interest includes wavelengths between 300 nm and 30 μm.

55. The method of claim 50, wherein the chemical compound of interest is a semiochemical.

56. The method of claim 55, wherein the semiochemical is at least one of a pheromone, a kairomone, an allomone, or a synomone.

57. The method of claim 50, wherein the chemical compound of interest is an odorant.

58. An apparatus comprising:
a power source;
a radiating source coupled to the power source, the radiating source being configured to emit an artificially simulated emission spectrum comprising at least one wavelength of a luminescing chemical compound of interest, the luminescing chemical compound of interest causing a behavioral response in an insect species.

59. The apparatus of claim 58, wherein at least one wavelength of the artificially simulated emission spectrum includes infrared radiation.

60. The apparatus of claim 58, wherein at least one wavelength of the artificially simulated emission spectrum includes visible radiation.

61. The apparatus of claim 58, wherein at least one wavelength of the artificially simulated emission spectrum includes ultraviolet radiation.

62. The apparatus of claim 58, wherein the artificially simulated emission spectrum simulates a natural emission spectrum of a luminescing compound of interest.

63. The apparatus of claim 58, wherein the chemical compound of interest is a semiochemical.

64. The apparatus of claim 63, wherein the semiochemical is at least one of a pheromone, a kairomone, an allomone, or a synomone.

65. The apparatus of claim 58, wherein the chemical compound of interest is an odorant.
